(19) United States
(12) Patent Application Publication
(10) Pub. No.: US 2023/0355882 A1
(43) Pub. Date: Nov. 9, 2023

(54) MEDICATION DELIVERY DEVICE WITH SENSING SYSTEM

(71) Applicant: Eli Lilly and Company, Indianapolis, IN (US)

(72) Inventors: Andrew Eric BOWYER, Indianapolis, IN (US); Joseph Edward KATUIN, Cicero, IN (US)

(21) Appl. No.: 18/040,282

(22) PCT Filed: Aug. 23, 2021
(86) PCT No.: PCT/US2021/047076

§ 371 Date: Feb. 2, 2023

Related U.S. Application Data
(60) Provisional application No. 63/070,465, filed on Aug. 26, 2020.

(51) Int. Cl. A61M 5/24; A61M 5/315

(52) U.S. Cl. CPC A61M 5/2422; A61M 5/31593; A61M 2205/50; A61M 2205/8206

(57) ABSTRACT

Medication delivery devices are provided having a sensor in the form of a single-pole double-throw (SPDT) switch, a conversion control module that receives signals from the SPDT switch and generates an outputs a signal, and related power control circuitry. The SPDT switch may interact with a rotating component having a plurality of teeth that slide against the arm during dose delivery. Contact of an arm of the SPDT switch to a peak of a tooth places the SPDT switch in a set state, while lack of contact between the arm and the peak of the tooth places the SPDT switch in a reset state. The SPDT switch and SR logic switch may be used to sense dosage of medication delivered during dose delivery. The power control circuitry can include a sleep state the reduces battery drain during non-use, and a wakeup state that senses the administered dose.

### BACKGROUND

Patients suffering from various diseases must frequently inject themselves with medication. To allow a person to conveniently and accurately self-administer medicine, a variety of devices broadly known as pen injectors or injection pens have been developed. Generally, these pens are equipped with a cartridge including a piston and containing a multi-dose quantity of liquid medication. A drive member is movable forward to advance the piston in the cartridge to dispense the contained medication from an outlet at the distal cartridge end, typically through a needle.

In disposable or prefilled pens, after a pen has been utilized to exhaust the supply of medication within the cartridge, a user discards the entire pen and begins using a new replacement pen. In reusable pens, after a pen has been utilized to exhaust the supply of medication within the cartridge, the pen is disassembled to allow replacement of the spent cartridge with a fresh cartridge, and then the pen is reassembled for its subsequent use.

Such devices may have components that physically interact with one another to result in a state change or an action by the device. For example, the device may have a cap that is removed prior to delivery, a dose button that may be rotated to set a dose and/or actuated to deliver a dose, an “on” button that wakes the device, and so on. Accordingly, the art has endeavored to provide reliable systems that accurately measure the relative movement of members of a medication delivery device in order to assess the dose delivered. Such systems may include a sensor which is secured to a first member of the medication delivery device and detects the relative movement of a sensed component secured to a second member of the device.

Many injector pens and other medication delivery devices do not include the functionality to automatically detect and record the amount of medication delivered by the device during the injection event. In the absence of an automated system, a patient must manually keep track of the amount and time of each injection. Accordingly, there is a need for a device that is operable to automatically detect information that can be correlated to the dose delivered by measuring mechanical parts of the medication delivery device during an injection event. There is also a need to improve the accuracy and reliability of the detection system.

### SUMMARY

The present disclosure relates to a medication delivery device having a sensor in the form of a single-pole double-throw (SPDT) switch, a conversion control module that receives signals from the SPDT switch and generates an output a signal, and related power control circuitry configured to preserve battery use when the medication delivery device is not administering a dose. The SPDT switch may interact with a mechanical component of the medication delivery device, such as a rotating component having a plurality of teeth that slide against an arm of the SPDT switch during dose delivery. Contact of the SPDT switch arm to a peak of a tooth places the SPDT switch in a set state, while lack of contact between the arm and the tooth places the SPDT switch in a reset state.

In some embodiments, the conversion control module comprises set/reset (SR) logic. The SR logic generates an undulating unit signal as the SPDT switch transitions between the set and reset states. The undulating unit signal may be used to sense dosage of medication delivered during dose delivery. According to some embodiments, a counting unit counts rising edges, falling edges, or both, of the undulating unit signal generated by the SR logic to determine an administered dose size.

The power control circuitry of the medication delivery device can include a sleep state and a wakeup state. The sleep state can reduce battery drain during non-use, and the wakeup state can fully power the conversion control module and related counting circuitry to sense the administered dose. According to some embodiments, the power control circuitry leverages a metal-oxide-semiconductor field-effect transistor (MOSFET) to prevent battery consumption when the medication delivery device is in a sleep state.

In one embodiment, a medication delivery device is provided. The medication delivery device includes: a housing; a mechanical switch mounted to a printed circuit board, wherein the mechanical switch comprises a single-pole double-throw (SPDT) switch comprising an arm; a rotatable element that is rotatable relative to the printed circuit board, the rotatable element having a series of protrusions that are spaced from one another, the rotatable element being positioned to permit the protrusions to slide against the arm of the SPDT switch; a conversion control module in electrical communication with the SPDT switch configured to generate an undulating unit signal based on signals from the SPDT switch as the arm slides against the protrusions; and a controller configured to receive the undulating unit signal from the conversion control module.

### BRIEF DESCRIPTION OF THE DRAWINGS

Additional embodiments of the disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1 is a perspective view of a medication delivery device having a dose detection system according to aspects of the present disclosure.

FIG. 2 is a partially exploded perspective view of the medication delivery device of FIG. 1, showing a dose button having a support and a cover, where the cover is shown separated from the support.

FIG. 3 is a partially exploded perspective view of the medication delivery device of FIG. 1 showing the components of the dose detection system.

FIG. 4 is a cross-sectional view of the medication delivery device of FIG. 1.

FIG. 5 is a partial cutaway view of a proximal end of the medication delivery device of FIG. 1, showing components of the dose detection system.

FIG. 6 is an underside view of a portion of the dose button of FIG. 1, showing a printed circuit board held within the dose button cover.

FIG. 7 is an exploded view of the portion of the dose button shown in FIG. 6.

FIG. 8 is a perspective view of a flange of a dose detection system of a medication delivery device.

FIG. 9 is a top down view of the flange of FIG. 8.

FIG. 10 is a perspective view of a dose button support.

FIG. 11 is a top down view of the dose button support of FIG. 10.

FIG. 12 shows an exemplary SPST switch, according to some examples.

FIGS. 13 and 14 show an exploded view of the electronics assembly and the flange of FIG. 5.

FIGS. 15-19 depict the cantilevered arm of the switch interacting with the rotating flange from FIGS. 8 and 9.

FIG. 20 is a diagram showing an exemplary waveform graph, showing the output current of a SPST switch over time.

FIG. 21 is a schematic of an exemplary conversion control module disposed between the dose detection system and a controller of a control system, according to some embodiments.

FIG. 22 is a schematic of a control signal generated by the conversion control module that is communicated to the controller of the control system, according to some embodiments.

FIGS. 23-24 are schematic diagrams of a microprocessor implementing SR logic in firmware, according to some embodiments.

FIG. 25 is an exemplary block diagram illustrating functional aspects of a printed circuit board for processing signals from a sensor, according to some embodiments.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended.

The present disclosure relates to sensing systems for medication delivery devices. In one aspect, the sensing system includes a single-pole double-throw (SPDT) switch in electrical communication with a conversion control module, such as set-reset (SR) logic. The sensing system further includes power control circuitry that can configure the medication delivery device to be in either a sleep state or a wakeup state based on the operation of the medication delivery device (e.g., whether the device is in use or not). A MOSFET is used to prevent battery consumption during the sleep state, as well as to provide full conversion control module operation in a wakeup state.

In some embodiments, the SPDT switch and processing circuitry (e.g., including the conversion control module and/or the power control circuitry) is used for sensing the relative rotational movement between a dose-setting assembly and an actuator of the medication delivery device in order to determine the amount of a dose delivered by a medication delivery device. The sensed relative rotational movements are correlated to the amount of the dose delivered. By way of illustration, the medication delivery device is described in the form of a pen injector. However, the medication delivery device may be any device which is used to set and to deliver a dose of a medication, such as pen injectors, infusion pumps and syringes. The medication may be any of a type that may be delivered by such a medication delivery device.

Such detection systems described herein may provide improved accuracy and reliability of determining the amount of rotation over other sensing systems that are arranged with contact event counters or other means, which are susceptible to signals with higher than desirable noise, such as signal debounce. Such detection systems may additionally, or alternatively, provide improved power consumption control compared to other sensing systems that are not configured to limit battery usage when the medication delivery device is not administering a dose. One of the advantages of use a mechanical switch, such as the SPDT switch, is the distance of travel of the sensed element for triggering between the on/off states can be smaller thereby providing greater resolution of sensing. In a mechanical switch with a conversion control module, once the switch activates the module, bounce of switch can be disregarded. In some applications, a mechanical switch may be preferred over other sensing, such as sliding contacts. For example, application of sliding contacts for sensing may be limited due the increasingly smaller size of circular travel and/or radial spacing of contact regions, making sensing in such smaller and tighter spaces much more difficult and less consistent. It may eventually result with less comparable resolution, such as, for example, the capability of only sensing every 36 degrees (or two units) instead of 18 degrees or less. Higher resolution of sensing the rotating sensed element can be achieved with a mechanical switch, such as SPDT switch, for a comparable geometry and size of the rotating sensed element. The teachings here could also apply to linear moving sensed elements and/or multiple single pole, single throw switches.

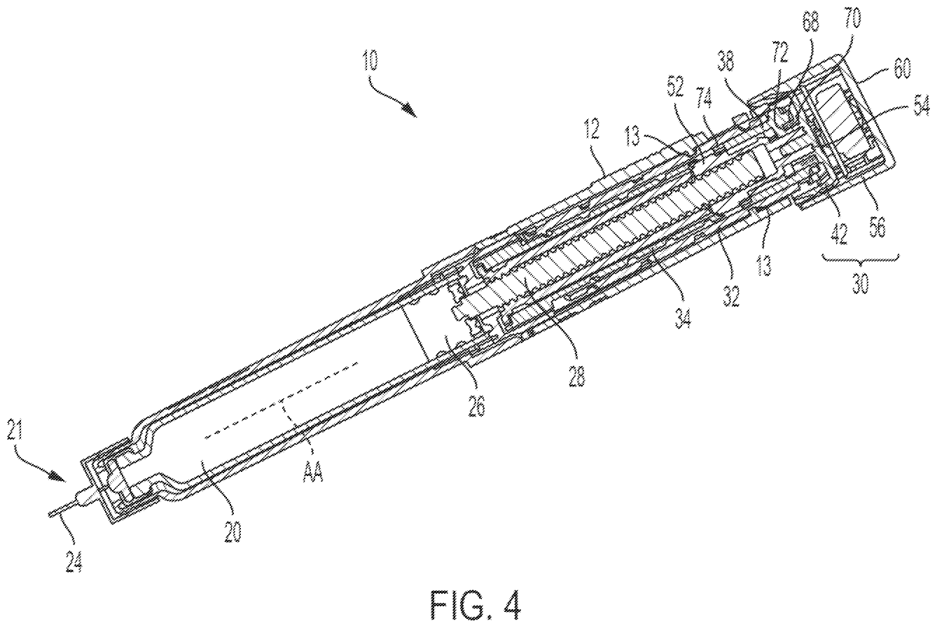

Devices described herein may further comprise a medication, such as for example, within a reservoir or cartridge 20 (see FIG. 4). In another embodiment, a system may comprise one or more devices including device 10 and a medication. The term “medication” refers to one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, oxyntomodulin analogs, oxyntomodulin derivatives, therapeutic antibodies and any therapeutic agent that is capable of delivery by the above device. The medication as used in the device may be formulated with one or more excipients. The device is operated in a manner generally as described above by a patient, caregiver or healthcare professional to deliver medication to a person.

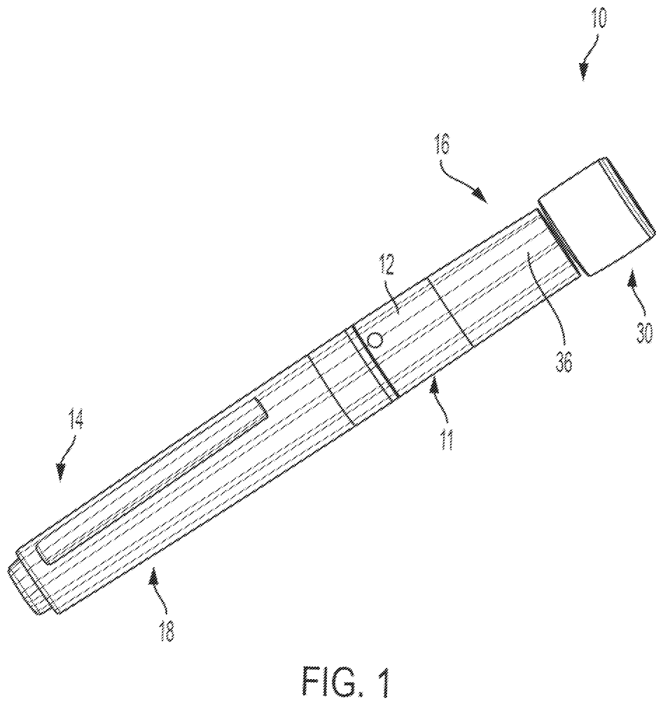

An exemplary medication delivery device 10 is illustrated in FIGS. 1-4 as a pen injector configured to inject a medication into a patient through a needle. Device 10 includes a body 11 that may comprise an elongated, pen-shaped housing 12 including a distal portion 14 and a proximal portion 16. Distal portion 14 may be received within a pen cap 18. Referring to FIG. 4, distal portion 14 may contain a reservoir or cartridge 20 configured to hold the medicinal fluid to be dispensed through the outlet 21 of the housing a dispensing operation. The outlet 21 of distal portion 14 may be equipped with an injection needle 24. In some embodiments, the injection needle is removable from the housing. In some embodiments, the injection needle is replaced with a new injection needle after each use.

A piston 26 may be positioned in reservoir 20. The medication delivery device may include an injecting mechanism positioned in proximal portion 16 that is operative to advance piston 26 toward the outlet of reservoir 20 during the dose dispensing operation to force the contained medicine through the needled end. The injecting mechanism may include a drive member 28, illustratively in the form of a screw, that is axially moveable relative to housing 12 to advance piston 26 through reservoir 20.

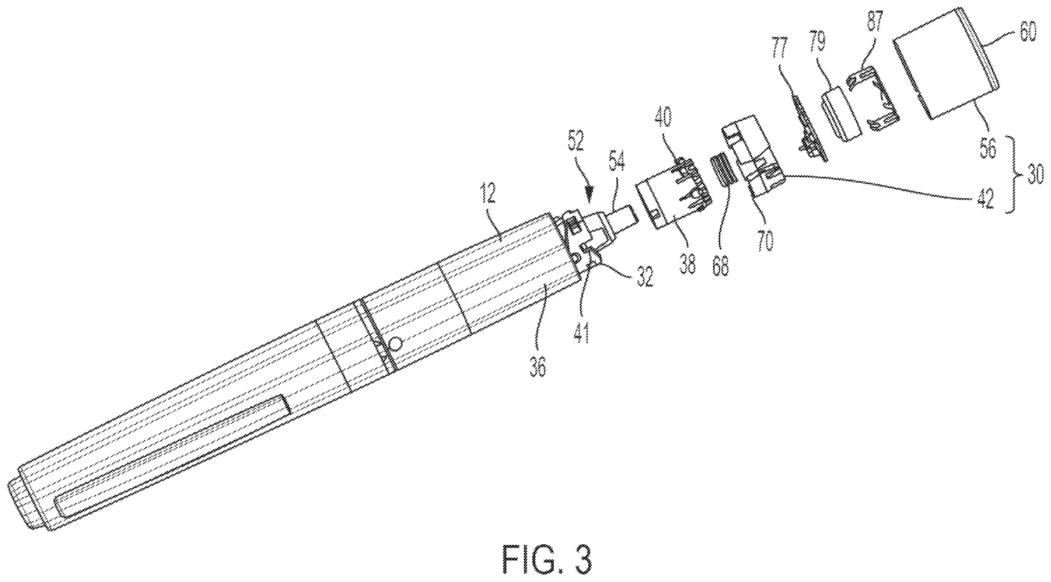

The device may include a dose-setting assembly coupled to the housing 12 for setting a dose amount to be dispensed by device 10. As best seen in FIGS. 3 and 4, in the illustrated embodiment, the dose-setting assembly includes a dose-setting screw 32 and a flange 38. The dose-setting screw 32 is in the form of a screw element operative to spiral (e.g., simultaneously move axially and rotationally) about a longitudinal axis AA of rotation relative to housing 12 during dose setting and dose dispensing. FIGS. 3 and 4 illustrate the dose-setting screw 32 fully screwed into housing 12 at its home or zero dose position. Dose-setting screw 32 is operative to screw out in a proximal direction from housing 12 until it reaches a fully extended position corresponding to a maximum dose deliverable by device 10 in a single injection. The extended positon may be any position between a position corresponding to an incremental extended position (such as a dose setting a 0.5 or 1 unit) to a fully extended position corresponding to a maximum dose deliverable by device 10 in a single injection and to screw into housing 12 in a distal direction until it reaches the home or zero position corresponding to a minimum dose deliverable by device 10 in a single injection.

Referring to FIGS. 3 and 4, dose-setting screw 32 includes a helically threaded outer surface that engages a corresponding threaded inner surface 13 of housing 12 to allow dose-setting screw 32 to spiral (e.g., simultaneously rotate and translate) relative to housing 12. Dose-setting screw 32 further includes a helically threaded inner surface that engages a threaded outer surface of sleeve 34 (FIG. 4) of device 10. The outer surface of dose-setting screw 32 includes dose indicator markings, such as numbers that are visible through a dosage window 36 to indicate to the user the set dose amount.

As mentioned above, in some embodiments, the dose-setting assembly further includes a tubular flange 38 that is coupled in the open proximal end of dose-setting screw 32 and is axially and rotationally locked to the dose-setting screw 32 by protrusions received within openings 41 in the dose-setting screw 32. The protrusions 40 of the flange 38 can be seen in FIGS. 3, 8 and 9, and the openings 41 of the dose-setting screw 32 can be seen in FIG. 3.

As seen in FIGS. 3 and 4, delivery device 10 may include an actuator assembly having a clutch 52 and a dose button 30. The clutch 52 is received within the dose-setting screw 32, and the clutch 52 includes an axially extending stem 54 at its proximal end. The dose button 30 of the actuator assembly is positioned proximally of the dose-setting screw 32 and flange 38. Dose button 30 includes a support 42, also referred to herein as an “under button,” and a cover 56, also referred to herein as an “over button.” As will be discussed, the support 42 and cover 56 enclose electronics components used to store and/or communicate data relating to amount of dose delivered by a medication delivery device.

The support 42 of the dose button may be attached to the stem 54 of the clutch 52, such as with an interference fit or an ultrasonic weld, so as to axially and rotatably fix together dose button 30 and clutch 52.

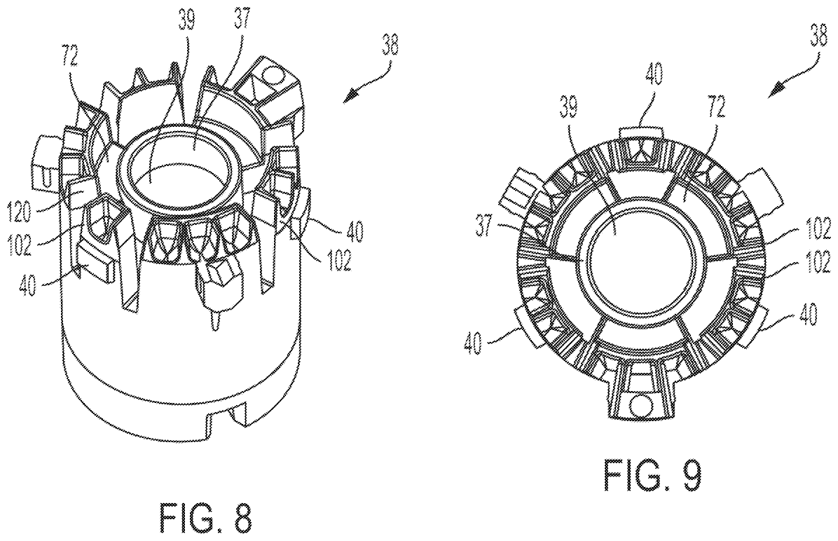

In some embodiments, a portion of the clutch may pass through a lumen 39 of the flange 38. The lumen 39 of the flange is best seen in FIGS. 8 and 9. The lumen 39 may, in some embodiments, serve to help center the clutch 52 in place.

Proximal face 60 of the dose button 30 may serve as a push surface against which a force can be applied manually, e.g., directly by the user to push the actuator assembly (dose button 30 and clutch 52) in a distal direction. A bias member 68, illustratively a spring, may be disposed between the distal surface 70 of support 42 and a proximal surface 72 of tubular flange 38 (FIGS. 8 and 9) to urge the support 42 of the actuation assembly and the flange 38 of the dose-setting assembly axially away from each other. Dose button 30 is depressible by a user to initiate the dose dispensing operation. In some embodiments, the bias member 68 is seated against this proximal surface 72 and may surround a raised collar 37 of the flange 38.

Delivery device 10 is operable in a dose setting mode and a dose dispensing mode. In the dose setting mode of operation, the dose button 30 is rotated relative to housing 12 to set a desired dose to be delivered by device 10. In some embodiments, rotating the dose button 30 in one direction relative to the housing 12 causes the dose button 30 to axially translate proximally relative to the housing 12, and rotating the dose button 30 in the opposite direction relative to the housing 12 causes the dose button 30 to axially translate distally relative to the housing. In some embodiments, clockwise rotation of the dose button moves the dose button 30 distally, and counter-clockwise rotation of the dose button moves the dose button proximally, or vice versa.

In some embodiments, rotating the dose button 30 to axially translate the dose button 30 in the proximal direction serves to increase the set dose, and rotating the dose button 30 to axially translate the dose button 30 in the distal direction serves to decrease the set dose. The dose button 30 is adjustable in pre-defined rotational increments corresponding to the minimum incremental increase or decrease of the set dose during the dose setting operation. The dose button may include a detent mechanism such that each rotational increment produces an audible and/or tactile “click.” For example, one increment or “click” may equal one-half or one unit of medication.

In some embodiments, the set dose amount may be visible to the user via the dial indicator markings shown through a dosage window 36. During the dose setting mode, the actuator assembly, which includes the which includes the dose button 30 and clutch 52, moves axially and rotationally with the dose-setting assembly, which includes the flange 38 and the dose-setting screw 32.

Dose-setting screw 32 and flange 38 are fixed rotationally to one another, and rotate and move proximally during dose setting, due to the threaded connection of the dose-setting screw 32 with housing 12. During this dose setting motion, the dose button is rotationally fixed relative to the flange 38 and the dose-setting screw 32 by complementary splines 74 of flange 38 and clutch 52 (FIG. 4), which are urged together by the bias member 68. In the course of dose setting, the dose-setting screw 32, flange 38, clutch 52, and dose button 30 move relative to the housing 12 in a spiral manner (e.g., simultaneous rotation and axial translation) from a “start” position to an “end” position. This rotation and translation relative to the housing is in proportion to the amount of dose set by operation of the medication delivery device 10.

Once the desired dose is set, device 10 is manipulated so the injection needle 24 properly penetrates, for example, a user's skin. The dose dispensing mode of operation is initiated in response to an axial distal force applied to the proximal face 60 of dose button 30. The axial force is applied by the user directly to dose button 30. This causes axial movement of the actuator assembly (dose button 30 and clutch 52) in the distal direction relative to housing 12.

The axial shifting motion of the actuator assembly compresses biasing member 68 and reduces or closes the gap between dose button 30 and the tubular flange 38. This relative axial movement separates the complementary splines 74 on clutch 52 and flange 38, and thereby disengages the dose button 30 from being rotationally fixed to the flange 38 and the dose-setting screw 32. In particular, the dose-setting screw 32 is rotationally uncoupled from the dose button 30 to allow backdriving rotation of the dose-setting screw 32 relative to the dose button 30 and the housing 12. Also, while the dose-setting screw 32 and flange 38 are free to rotate relative to the housing 12, the dose button 30 is held from rotating relative to the housing 12 by the user's engagement of dose button 30 by pressing against it.

As dose button 30 and clutch 52 are continued to be axially plunged without rotation relative to housing 12, dose-setting screw 32 screws back into housing 12 as it spins relative to dose button 30. The dose markings that indicate the amount still remaining to be injected are visible through window 36. As dose-setting screw 32 screws down distally, drive member 28 is advanced distally to push piston 26 through reservoir and expel medication through needle 24.

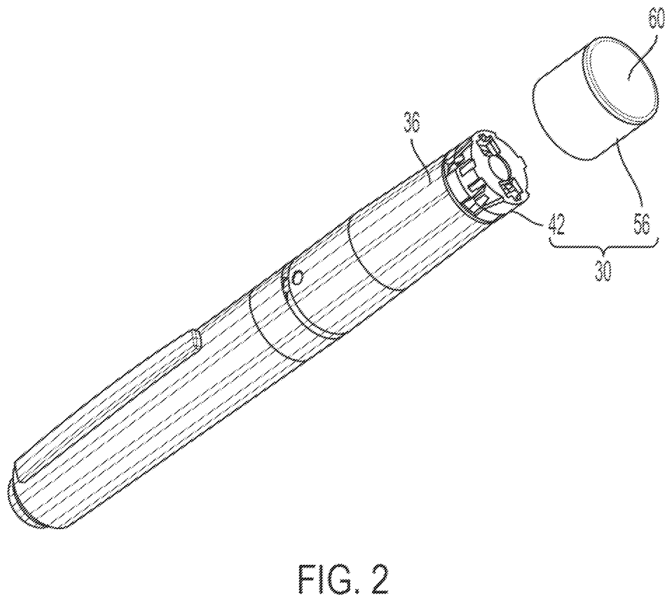

During the dose dispensing operation, the amount of medicine expelled from the medication delivery device is proportional to the amount of rotational movement of the dose-setting screw 32 relative to the housing 12 as the dose-setting screw 32 screws back into housing 12. In some embodiments, because the dose button 30 is rotationally fixed relative to the housing 12 during the dose dispensing mode, the amount of medicine expelled from the medication delivery device may be viewed as being proportional to the amount of rotational movement of the dose-setting screw 32 relative to the dose button 30 as the dose-setting 32 screws back into housing 12. The injection is completed when the internal threading of dose-setting screw 32 has reached the distal end of the corresponding outer threading of sleeve 34 (FIG. 4). Device 10 is then once again arranged in a ready state or zero dose position as shown in FIGS. 2 and 4.

As discussed above, the dose delivered may be derived based on the amount of rotation of the dose-setting assembly (flange 38 and dose-setting screw 32) relative to the actuator assembly (clutch 52 and dose button 30) during dose delivery. This rotation may be determined by detecting the incremental movements of the dose-setting assembly which are “counted” as the dose-setting assembly is rotated during dose delivery.

Further details of the design and operation of an exemplary delivery device 10 may be found in U.S. Pat. No. 7,291,132, entitled Medication Dispensing Apparatus with Triple Screw Threads for Mechanical Advantage, the entire disclosure of which is hereby incorporated by reference herein. Another example of the delivery device is an auto-injector device that may be found in U.S. Pat. No. 8,734,394, entitled “Automatic Injection Device With Delay Mechanism Including Dual Functioning Biasing Member,” which is hereby incorporated by reference in its entirety, where such device being modified with one or more various sensor systems described herein to determine an amount of medication delivered from the medication delivery device based on the sensing of relative rotation within the medication delivery device. Another example of the delivery device is a reusable pen device that may be found in U.S. Pat. No. 7,195,616, entitled “Medication Injector Apparatus with Drive Assembly that Facilitates Reset,” which is hereby incorporated by reference in its entirety, where such device being modified with one or more various sensor systems described herein to determine an amount of medication delivered from the medication delivery device based on the sensing of relative rotation within the medication delivery device.

Described herein is a dose detection system that may be operable to determine the amount of dose delivered based on relative rotation between a dose setting member and the device body. The dose detection system utilizes a dose setting member attached to the device body and rotatable relative to the device body about an axis of rotation during dose delivery. A sensed element is attached to and rotationally fixed with the dose setting member. An actuator is attached to the device body and is held against rotation relative to the device body during dose delivery. The sensed element thereby rotates relative to the actuator during dose delivery in relation to the amount of dose delivered.

In some embodiments, the dose detection system comprises a rotational sensor attached to the actuator assembly and a sensed element that includes surface features that are equally radially spaced about the axis of rotation of the sensed element.

In some embodiments, the dose detection systems may include a sensor and a sensed component attached to components of the medication delivery device. The term “attached” encompasses any manner of securing the position of a component to another component or to a member of the medication delivery device such that they are operable as described herein. For example, a sensor may be attached to a component of the medication delivery device by being directly positioned on, received within, integral with, or otherwise connected to, the component. Connections may include, for example, connections formed by frictional engagement, splines, a snap or press fit, sonic welding or adhesive.

The term “directly attached” is used to describe an attachment in which two components, or a component and a member, are physically secured together with no intermediate member, other than attachment components. An attachment component may comprise a fastener, adapter or other part of a fastening system, such as a compressible membrane interposed between the two components to facilitate the attachment. A “direct attachment” is distinguished from attachment where the components/members are coupled by one or more intermediate functional members.

The term “fixed” is used to denote that an indicated movement either can or cannot occur. For example, a first member is “fixed rotationally” with a second member if the two members are required to move together in rotation. In one aspect, a member may be “fixed” relative to another member functionally, rather than structurally. For example, a member may be pressed against another member such that the frictional engagement between the two members fixes them together rotationally, while the two members may not be fixed together absent the pressing of the first member.

Various sensor arrangements are contemplated herein. In general, the sensor arrangements comprise a sensor and a sensed component. The term “sensor” refers to any component which is able to detect the relative position or movement of the sensed component. The sensor may be used with associated electrical components to operate the sensor. The “sensed component” is any component for which the sensor is able to detect the position and/or movement of the sensed component relative to the sensor. For the dose detection system, the sensed component rotates relative to the sensor, which is able to detect the rotational movement of the sensed component. The sensor may comprise one or more sensing elements, and the sensed component may comprise one or more sensed elements. The sensor detects the movement of the sensed component and provides outputs representative of the movement of the sensed component.

Illustratively, the dose detection system includes an electronics assembly suitable for operation of the sensor arrangement as described herein. The medication delivery device may include a controller that is operably connected to the sensor to receive outputs from the sensor. The controller begins receiving generated signals from the sensor indicative of counts from first to last one for a total number of counts that is used for determining total displacement, e.g. angular displacement. In the case of detecting an angular movement of a dose-setting assembly, the controller may be configured to receive data indicative of the angular movement of the dose-setting assembly that can be used to determine from the outputs the amount of dose delivered by operation of the medication delivery device. The controller may be configured to determine from the outputs the amount of dose delivered by operation of the medication delivery device. The controller may include conventional components such as a processor, power supply, memory, microcontrollers, etc. Alternatively, at least some components may be provided separately, such as by means of a computer, smart phone or other device. Means are then provided to operably connect the external controller components with the sensor at appropriate times, such as by a wired or wireless connection.

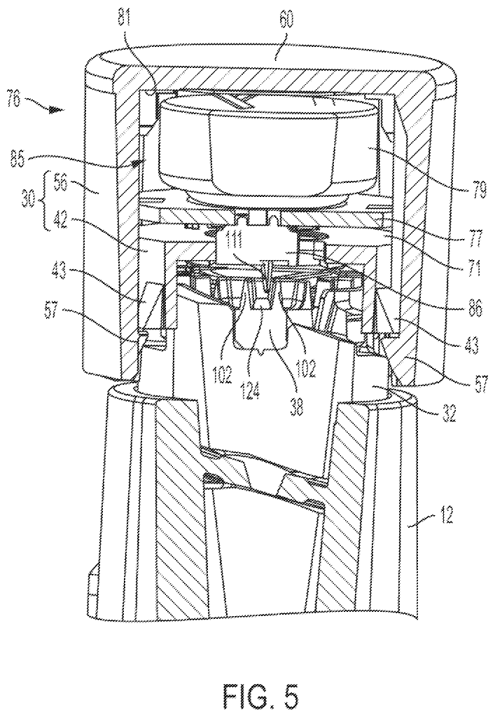

According to one aspect, the electronics assembly includes a sensor arrangement including one or more sensors operatively communicating with a processor for receiving signals from the sensor representative of the sensed rotation. An exemplary electronics assembly 76 is shown in FIGS. 5-7 and can include a sensor 86, and a printed circuit board (PCB) 77 having a plurality of electronic components. The printed circuit board may be a flexible printed circuit board. The circuit board of the electronics assembly 76 may include a microcontroller unit (MCU) as the controller comprising at least one processing core and internal memory. The electronics assembly may include a power source 79, e.g. a battery, illustratively a coin cell battery, for powering the components. The controller of electronics assembly 76 may include control logic operative to perform the operations described herein, including detecting the angular movement of the dose-setting assembly during dose setting and/or dose delivery and/or detecting a dose delivered by medication delivery device 10 based on a detected rotation of the dose-setting assembly relative to the actuator assembly. Many, if not all of the components of the electronics assembly, may be contained in a compartment 85 within the dose button 30. In some embodiments, the compartment 85 may be defined between a proximal surface 71 of support 42 of the dose button and a distal surface 81 of the cover 56 of the dose button. In the embodiment shown in FIG. 5, the electronics assembly 76 is permanently integrated within the dose button 30 of the delivery device. In other embodiments, the electronics assembly is provided as a module that can be removably attached to the actuator assembly of the medication delivery device.

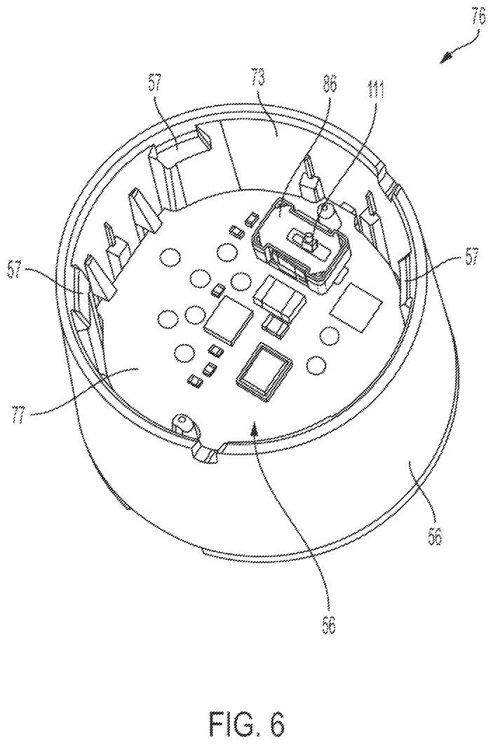

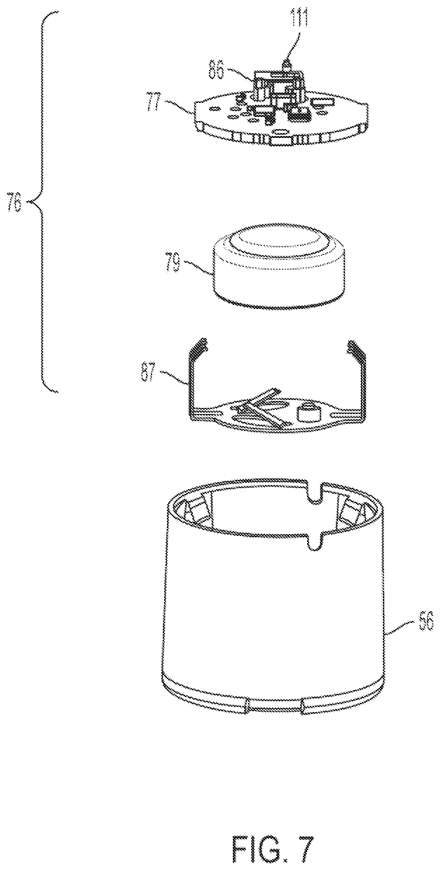

An underside view of the electronics assembly 76 held within the cover 56 is shown in FIG. 6, and an exploded view of the electronics assembly 76 is shown in FIG. 7. As shown in FIGS. 6 and 7, the electronics assembly 76 may include a printed circuit board (PCB) 77 and a sensor 86 having a contact surface 111. As shown in FIG. 7, the electronics assembly 76 may also include a battery 79 and a battery cage 87.

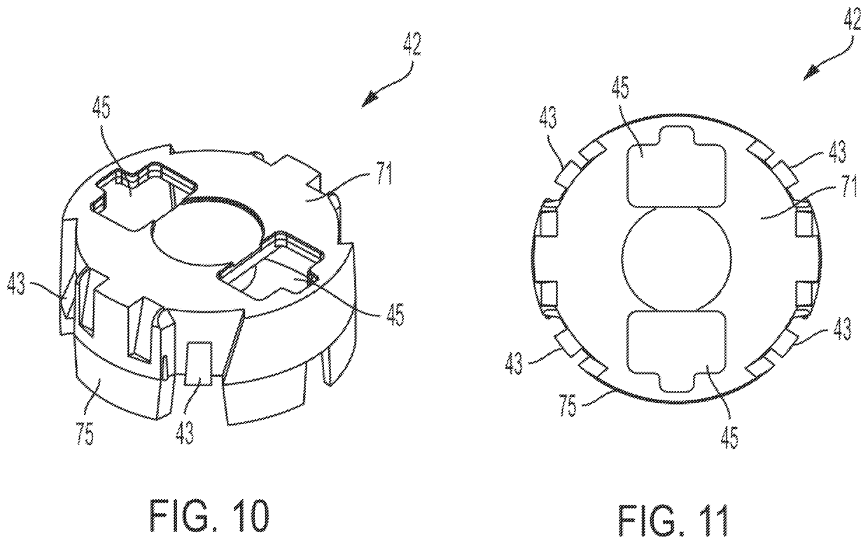

In some embodiments, at least a portion of the sensor 86 extends out of the compartment 85 of the dose button 30. As best seen in FIGS. 10 and 11, the support 42 of the dose button 30 may include one or more openings 45 through which the sensor 86 can extend through. In some embodiments, during assembly of the medication delivery device, the contact surface 111 of the sensor 86 is passed through the opening 45 of the support 42. This may permit the contact surface 111 of the sensor to interact with a component that is external to the compartment 85 of the dose button 30. In some embodiments, while only one of the openings 45 in the support 42 is needed to accommodate a sensor, a second opening may be provided, e.g. for symmetry of the support component, which help with manufacturing of the component and/or assembly of the component with the medication delivery device.

The controller of electronics assembly 76 may be operative to store the total angular movement used for determining dose delivery and/or the detected dose delivery in local memory (e.g., internal flash memory or on-board EEPROM). The controller may be further operative to wirelessly transmit a signal representative of the total counts, total angular movement, and/or detected dose to an external device, such as a user's mobile device or a remote server. Transmission may, for example, be over a Bluetooth low energy (BLE) or other suitable short or long range wireless communication protocol. Illustratively, the BLE control logic and controller are integrated on the same circuit.

As discussed, according to one aspect, the dose detection system involves detecting relative rotational movement between two assemblies of the medication delivery device. With the extent of rotation having a known relationship to the amount of a delivered dose, the sensor operates to detect the amount of angular movement from the start of a dose injection to the end of the dose injection. For example, in some embodiments, the relationship for a pen injector is that an angular displacement of a dose-setting assembly of 18° is the equivalent of one unit of dose, although other angular relationships are also suitable, such as, for example, 9, 10, 15, 20, 24 or 36 degrees may be used for a unit or a half unit. The sensor system is operable to determine the total angular displacement of a dose setting member during dose delivery. Thus, if the angular displacement is 90°, then 5 units of dose have been delivered.

The angular displacement is determined by counting increments of dose amounts as the injection proceeds. For example, a sensing system may use a repeating pattern of a sensed element, such that each repetition is an indication of a predetermined degree of angular rotation. Conveniently, the pattern may be established such that each repetition corresponds to the minimum increment of dose that can be set with the medication delivery device.

The dose detection system components may be permanently or removably attached to the medication delivery device. In some embodiments, at least some of the dose detection system components are provided in the form of a module that is removably attached to the medication delivery device. In other embodiments, the dose detection system components are permanently attached to the medication delivery device.

In some embodiments, a sensor may detect, during dose delivery, the relative rotation of a sensed component that is rotationally fixed to the dose-setting screw 32, from which is determined the amount of a dose delivered by the medication delivery device. In an illustrative embodiment, a rotational sensor is attached, and rotationally fixed, to the actuator assembly. The actuator assembly does not rotate relative to the device housing during dose delivery.

In some embodiments, a sensed component is attached, and rotationally fixed, to the dose-setting screw 32, which rotates relative to the dose button 30 and the device housing 12 during dose delivery. In some of the embodiments described herein, the sensed component includes a ring structure having a plurality of proximally extending projections circumferentially disposed relative to one another. Projections are shaped and sized to deflect a movable element of the rotational sensor. One illustrative embodiment of such a sensed component is tubular flange 38, best seen in FIGS. 3, 5, 8, and 9. Embodiments described herein may be provided for a module that is removably attachable to the dose button of the delivery device or integrated within the dose button of the delivery device.

During dose delivery, dose-setting screw 32 is free to rotate relative to dose button 30. In the illustrative embodiment, the electronics assembly 76 is rotationally fixed with the dose button 30 and does not rotate during dose delivery.

As seen in FIGS. 2, 3 and 5, the dose button 30 comprises a cover 56 coupled to a support 42. An electronics assembly 76 may be at least partially contained within a compartment 85 defined between the cover 56 and the support. In some embodiments, the cover and support have corresponding splines that engage with one another to couple the cover and support together. For example, in some embodiments, the cover 56 may couple to the support 42 via one or more snaps 57 on the cover 56 and corresponding to one or more protrusions 43 on the support. As seen in FIGS. 5 and 6, the snaps 57 on the cover 56 may be directed radially inwardly from an inner circumferential sidewall 73. As seen in FIGS. 5, 10 and 11, the protrusions 43 on the support 42 may be directed radially outwardly from an outer circumferential sidewall 75 of the support 42. The protrusions 43 may form a triangular ramp shape.

The snaps 57 on the cover 56 are configured to snap over and mate with the protrusions 43 on the support to couple the cover to the support. In some embodiments, the protrusion on the support comprises a continuous annular protrusion around the outer circumferential sidewall of the support. The cover 56 may attach to the support 42 via frictional engagement, interference fit or any other suitable fit. In some embodiments, the cover 56 is permanently fixed to the support 42 during assembly, e.g. via ultrasonic welding, adhesive, or other suitable fixation approach.

As seen in FIGS. 8 and 9, the tubular flange 38 may include a plurality of axially directed teeth 102 that are equally radially spaced about a rotation axis and arranged to correlate to the equivalent of one unit of dose. In this illustrative embodiment, the tubular flange 38 includes 20 teeth 102 that are equally rotationally spaced from one another, such that the rotation distance between two adjacent teeth corresponds to 18 degrees of rotation. Thus, with the tubular flange 38 of FIG. 8, 18 degrees of rotation of the tubular flange 38 may be used to represent one dosage unit or a half dosage unit. It should be appreciated that, in other embodiments, different total numbers of teeth may be used to create other angular relationships, such as, for example, 9, 10, 15, 18, 20, 24 or 36 degrees may be used for a unit or 0.5 unit.

A recess 124 may be defined between each pair of adjacent teeth 102. Each tooth 102 may have an approximately triangular shaped profile, each having a surface 120 against which a contact surface 111 of a sensor may slide.

In some embodiments, the sensor for detecting rotation of the tubular flange includes a movable element that has a contact portion capable of resting against the teeth of the tubular flange and is spring-biased such that the contact surface is configured to slide against and over the teeth during rotation of the flange relative to the actuator assembly during dose delivery. The sensor is responsive to the movement of the contact portion over the teeth and generates signals corresponding to the flange. A controller is responsive to the signals generated by the sensor to determine a dose count for determining the dosage delivered based on the detected rotation of the flange relative to the actuator assembly during dose delivery.

The contact surface may be biased against the physical features of the tubular flange to ensure proper contact between the contact surface and the physical features during rotation. In one embodiment, the movable element is a resilient member having one portion attached to the actuator at a location displaced from the contact surface. In one example, the movable element is a following member comprising a beam attached at one end to the actuator and having the contact surface at the other end. The beam is flexed to urge the contact surface in the direction of the surface features. Alternatively, the movable element may be biased in any of a variety of other ways. In addition to the use of a resilient beam, the biasing may be provided, for example, by use of a spring component. Such spring component may for example comprise a compression, tension, or torsion coil spring. In yet other embodiments, the movable element may be biased against the surface features of the sensed element by a separate resilient member or spring component bearing against the movable element.

FIG. 5 depicts an illustrative embodiment of a sensor 86 having a contact surface 111 interacting with teeth 102 of a tubular flange 38. As the flange 38 rotates relative to the dose button 30 during delivery, the teeth 102 of the flange contact and slide against the contact surface 111 of the sensor 86, causing the contact surface 111 to move in an oscillating manner. The movement of the contact surface 111 may be a combination of axial and lateral movement as the contact surface 111 slides into and out of the recesses 124 defined between the teeth 102 of the flange 38. The sensor 86 may be configured to track the movement of the contact surface 111 and associate the movement with an output signal that is sent to a controller.

As alternative to teeth on the tubular flange, surface features that interact with the sensor may comprise anything detectable by the sensor. The sensor arrangement may be based on a variety of sensed characteristics, including tactile, optical, electrical and magnetic properties, for example. In the illustrative embodiments shown in the figures, the surface features are physical features which allow for detection of incremental movements as the dose-setting assembly rotates relative to the actuator assembly. In alternative embodiments, the sensor may be a piezoelectric sensor, a magnetic sensor such as a Hall effect sensor, an accelerometer for detecting vibration, e.g. of a ratcheting or other detent mechanism, where vibration can be correlated with rotational movement, an optical sensor such as a reflective sensor, an interrupter sensor, or an optical encoder, or any other sensor suitable for sensing rotation of a first component relative to a second component.

In some embodiments, when a user presses axially on face 60 of the dose button 30, the dose button 30 advances distally relative to the housing 12, compressing spring 68. Continued pressing of the dose button 30 distally results in back driving of the dose-setting screw 32 in a spiral direction relative to housing 12. As a result, the dose-setting screw 32 and flange 38 are driven to rotate by the axially pressing upon the dose button 30. In some embodiments, the dose detection system is operable for dose detection only while the dose button is being pressed.

In some embodiments, the electronics assembly may include a clock or timer to determine the time elapsed between counts caused by trigger of the rotational sensor from the surface features of the sensed element. When no counts have been detected by the controller after a period of time this may be used to indicate that the dose has completed.

In some embodiments, a single sensing system may be employed for both dose detection sensing and wake-up activation. For example, upon the initial sensing of rotation of the sensed element by the sensor, the controller is configured to allow wake-up or activation of the electronics assembly to a greater or full power state. The wake-up feature is configured to allow power transmission from the power source (shown as battery) for powering up the electronic components for dose sensing in order to minimize inadvertent power loss or usage when a dose dispensing event is not occurring. In other embodiments, a separate wake-up switch may be provided and arranged within the dose button housing and triggered when the dose button is in its distal position. After activation of the electronics assembly, the controller begins receiving generated signals from the rotational sensor indicative of counts from first to last one for a total number of counts that is used for determining total angular displacement and thus the amount of dose delivered.

In some embodiments, the electronics assembly may have a controller that is configured to receive an output signal from a rotational sensor. The controller of the electronics assembly may be programmed to convert the intermediate signal to a conditioned digital signal, which may be a single step/square wave with a predetermined width representing a predetermined time. In some embodiments, output signals that are less than a predetermined level may be filtered out and ignored.

According to one aspect, a medication delivery device includes a repeatedly activatable switch that may serve as a sensor. In some embodiments, the switch serves as the rotational sensor in the dose detection system described above. In other embodiments, however, the switch may be used to detect other activity such as removal of a cap.

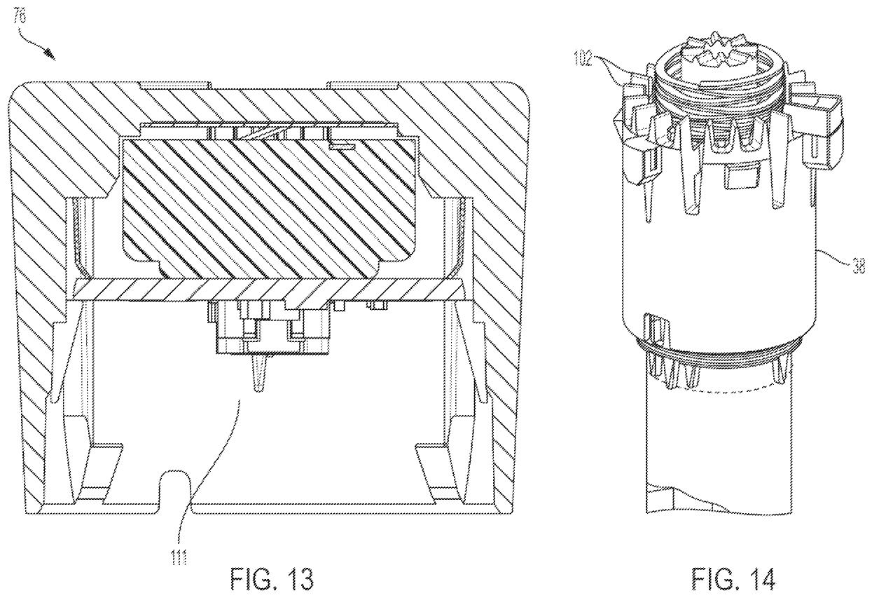

As discussed herein, the electronics assembly includes a sensor arrangement with one or more sensors operatively communicating with a processor to receive signals from the sensor that are representative of the sensed rotation of the sensed member. FIGS. 13 and 14 show an exploded view of the electronics assembly 76 and the flange 38 of FIG. 5. FIG. 13 shows the contact surface 111 that interacts with the teeth 102 of the tubular flange 38. As the flange 38 rotates relative to the dose button during delivery, the teeth 102 of the flange contact and slide against the contact surface 111 of the sensor, causing the contact surface 111 to move in an oscillating manner.

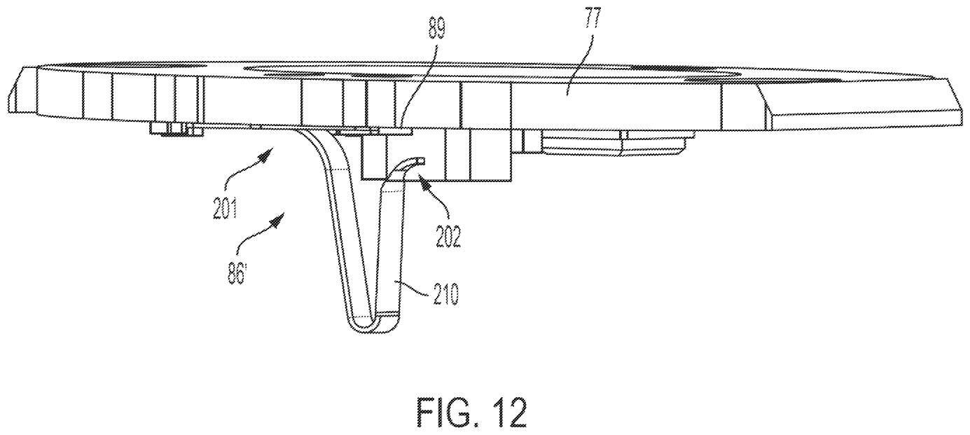

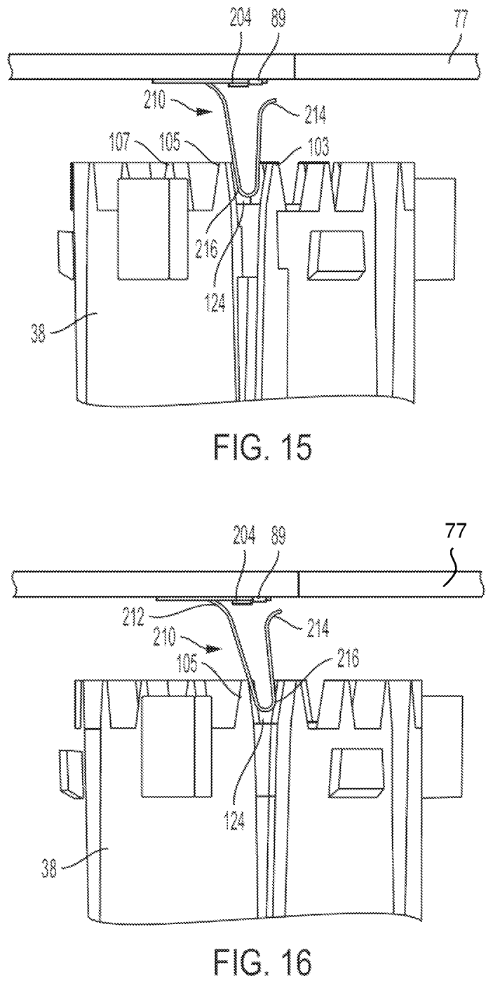

Various sensor arrangements can be employed for medication delivery devices. Exemplary sensor arrangements include mechanical components (e.g., sliding contacts, switches), piezoelectric components, and/or the like. Some exemplary techniques include a mechanical switch that is triggered on the teeth of the flange. For example, a single pole, single throw (SPST) switch can be used to sense rotation of the flange as the teeth slide against the switch. For example, a SPST switch can be implemented using a switch rocker arm as shown in FIG. 12 or, such as, for example, the switch in FIG. 13, which depicts a switch 86′ having a conductive pad 89 and a cantilevered arm 210. The conductive pad 89 and a first end 201 of the cantilevered arm 210 are mounted to a PCB 77. The switch also includes a base that is connected to the cantilevered arm 210. The base is connected to the PCB to connect cantilevered arm to the PCB. The base and the arm together may form a single monolithic component. FIGS. 15-19 depict the cantilevered arm 210 of the switch interacting with the rotating flange 38 from FIGS. 8 and 9. FIG. 15 shows the arm 210 in an unstressed state, as the third curved portion 216 is situated within a recess 124 between two adjacent teeth 103, 105. In FIG. 16, the flange 38 has begun to rotate relative to the switch and the PCB 77. As a result, tooth 105 slides and pushes against the third curved portion 216 of the arm 210, causing the arm 210 to begin to deflect toward a direction out of the recess 124. The first curved portion 212 begins to move toward a straightened configuration, and the second curved portion 214 begins to move toward the conductive pad 89.

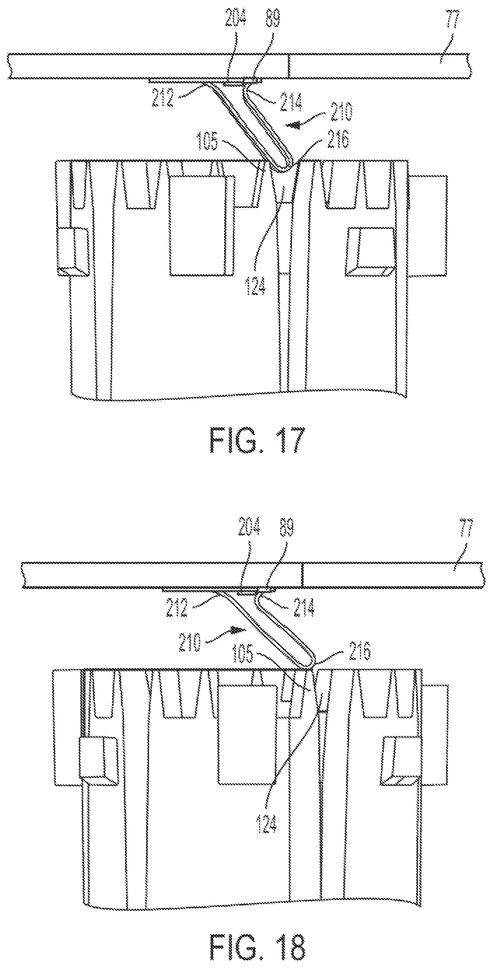

In FIG. 17, the flange 38 has rotated further than in FIG. 16, causing the tooth 105 to slide against and push the third curved portion 216 nearly completely out of the recess 124. The first curved portion 212 has moved even more toward a straightened configuration. As a result, the second curved portion 214 has made contact with the conductive pad 89, thereby closing the switch. The second curved portion also is pressed against a blocking protrusion 204, which prevents the second curved portion from moving further toward the first curved portion 212, and may help to prevent the second curved portion from bouncing repeatedly against the conductive pad 89 in a rapid manner that may give rise to a noisy output signal.

In FIG. 18, the flange 38 has rotated further than in FIG. 17, and the third curved portion 216 has exited the recess 124 and is sliding across the top of tooth 105. The second curved portion 214 remains in contact with both the conductive pad 89 and the blocking protrusion 204. Blocking protrusion 204 has prevented the second curved portion 214 from moving closer to the first curved portion 212.

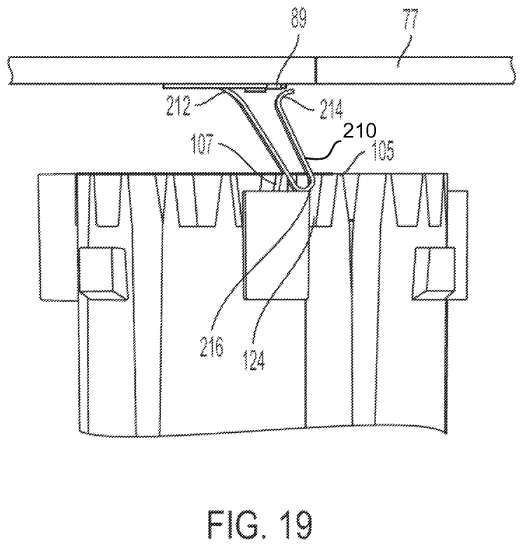

Finally, in FIG. 19, the flange 38 has rotated further than in FIG. 18, and the third curved portion 216 has stopped contacting tooth 105 and has now begun contacting the next adjacent tooth, 107. During this transition as the next tooth 107 is just beginning to push upon the arm 210, the arm, which is spring biased toward the position shown in FIG. 15, has swung back toward its unstressed state, thus causing the first curved portion 212 to move toward a more curved shape, resulting in movement of the third curved portion 216 toward a direction opposite to the rotation direction of the flange 38 and resulting in movement of the second curved portion 214 away from the conductive pad 84, thereby opening the switch. As the flange 38 rotates further, the cycle continues and the arm moves back toward the conductive pad to close the switch, and so on.

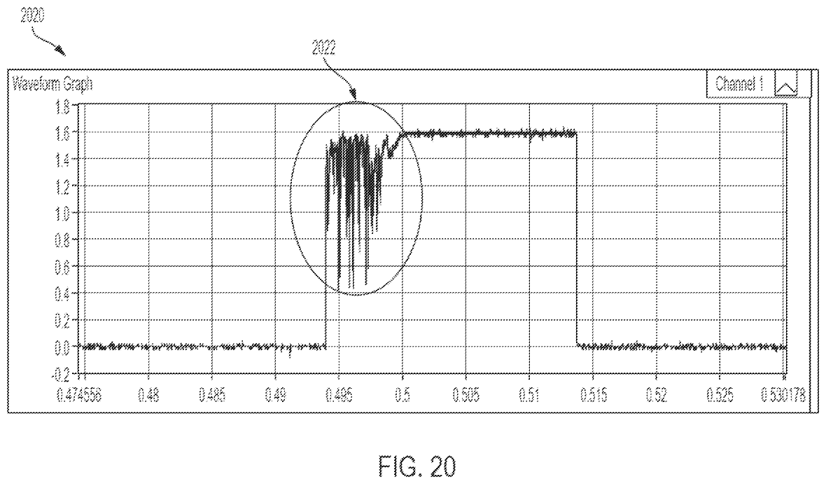

As described herein, switches can close and open based on mechanical interaction with the teeth of the flange of the medication delivery device, such that as the switch passes across the teeth, signals are sent to processing circuitry (e.g., to a general purpose input/output (GPIO) of a microprocessor of the medication delivery device). Over time use of the medication delivery device can cause the components to scratch, which can affect the mechanical operation of the switch. Additionally, or alternatively, during use of the medication delivery device, the opening and/or closing current of the switch can bounce. FIG. 20 is a diagram showing an exemplary waveform graph 2000, showing the output current of a SPST switch over time. The output current is approximately zero (0) when the switch is open, and approximately 1.6 volts when the switch is closed. As can be seen in the portion 2022 of the graph 2000, the electrical bouncing of the switch between open and closed causes the output current to fluctuate, which can in-turn be interpreted as a number of different trigger signals, when in actuality the switch is only closing once. The bounce can be further complicated due to the switch dimensions. For example, such switches can be light switches, such that during transitions a high impedance can cause the switch to open and/or close quickly (e.g., in microseconds). Such bouncing transitions may appear to a microprocessors as a plurality of pulses when the measurement is in actuality just for one switch transition or pulse. Therefore, conventional sensor arrangements can suffer from one or more deficiencies, including due to the materials and/or electrical properties of the sensor, which can cause errors in the dose counts or dose measurements calculated based on the sensor output.

According to some embodiments, the techniques disclosed herein can provide for using a single pole, double throw (SPDT) switch as the sensing mechanism and software and/or hardware-based signal processing configured to provide low-error signals to the microprocessor of the medication delivery device. According to some embodiments, the SPDT switch can include set and reset states that can be processed using a conversion control module (e.g., set/reset (SR) flip flop logic) to drive high and low logic levels to downstream circuitry, such as the microcontroller (e.g., via a general purpose input/output (GPIO) of the microcontroller). Such a SPDT switch and associated logic can address one or more deficiencies of conventional sensor arrangements. For example, some embodiments can reduce and/or eliminate signal bounce compared to that caused by conventional sensor techniques. As another example, the techniques can leverage a circuit design as described herein to provide for low power consumption when the medication delivery device is not in use.

In some embodiments, the SPDT switch comprises a cantilevered arm that is moveable to place the SPDT switch into a plurality of states. For example, the SPDT switch can include a set state, a reset state, and/or a neutral state. The cantilevered arm may be mounted to a printed circuit board at a first end, and a second end of the arm may be unattached and free to move relative to the printed circuit board.

In some embodiments, the set state and reset state may be associated with respective conductive pads that are mounted to the PCB and/or to a housing device that is in-turn mounted to the PCB. Contact between the cantilevered arm and a conductive pad closes the switch for that associated state, while lack of contact between either or both pads opens the switch.

In some embodiments, the cantilevered arm may be configured to contact and slide against the sensed component, e.g. against the teeth of a rotating tubular flange 38 shown in FIGS. 8 and 9. In some embodiments, contact of the SPDT switch arm to a portion of a tooth (e.g., to a peak of a tooth) places the SPDT switch in one state (e.g., a set state), while contact of the SPDT switch arm to a second portion of a tooth and/or lack of contact between the arm and the tooth places the SPDT switch in a second state (e.g., a reset state).

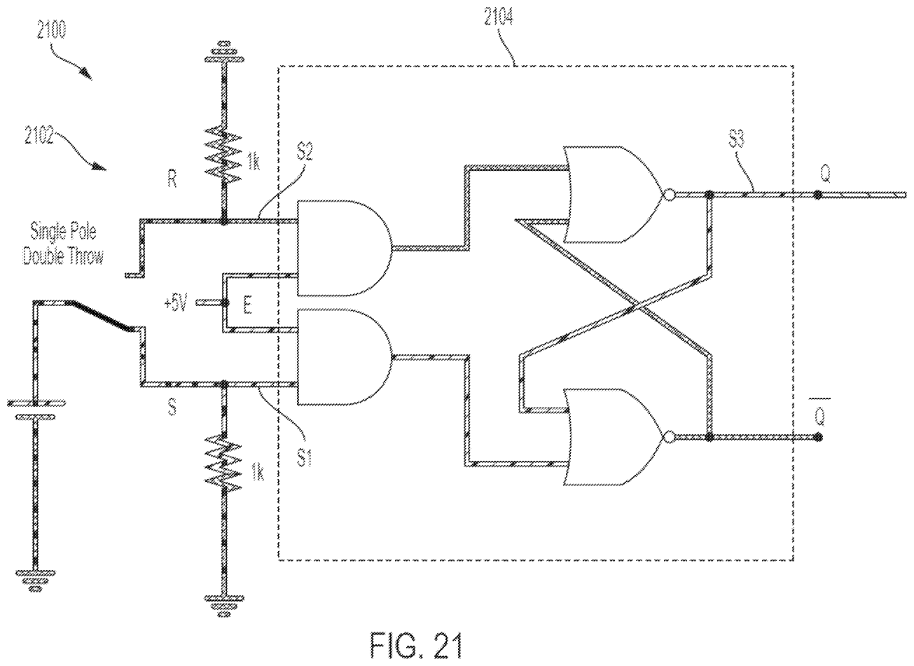

In some embodiments, a conversion control module may be disposed between the sensor and the processing core of the MCU. In some embodiments, the conversion control module can be implemented by the microprocessor (e.g., in firmware and/or software). As described further in FIG. 21, according to some embodiments the conversion control module is configured to generate an undulating unit signal S3 from the generated first and second signals S1, S2 (that are in an alternating arrangement), which may also be referred to as the set signal S and reset signal R, respectively.

In some embodiments, the conversion control module comprises a latch circuit, a SR latch circuit, and/or the like. The latch circuit can include an output signal that will toggle high or low depending on alternating contact input signals received by the latch circuit. The conversion control module is operable to convert the first and second signals S1, S2 shown in FIG. 21 into a switch-like, GPIO signal as a single input to the processing core of the MCU. One of the potential benefits of providing a latch circuit is that the processing power demand may be reduced compared to other configurations.

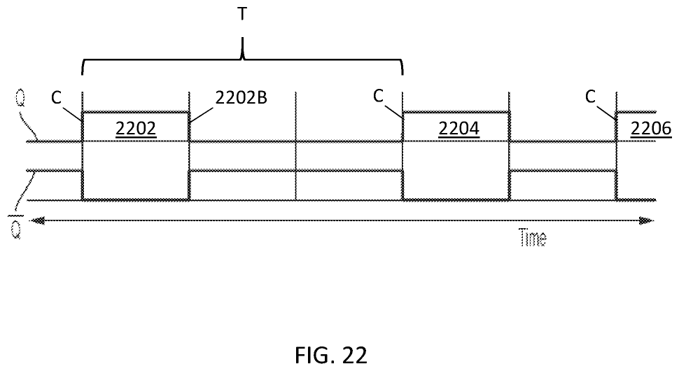

FIG. 21 illustratively depicts an example of a system 2100 with a SPDT switch 2102 and an SR latch circuit 2104. While not shown, in some embodiments as described herein the SR latch circuit 2104 may be disposed between the SPDT switch 2102 and the processing core of the controller MCU (e.g., which is in electrical communication with the Q signal). In some embodiments, the SR latch circuit 2104 may be implemented partially and/or entirely by the processing core of the MCU. The SPDT switch 2102 generates the sensing signal. Each throw of the SPDT switch 2102 is associated with a corresponding set S circuit or reset R circuit. The latch circuit 2104 of the conversion control module is shown to receive the reset signal R, shown as S2, and the set S signal, shown as S1, and flip-flop between set and reset to generate Q and not-Q signals. An example of the Q and not-Q signals is shown in FIG. 22.

In some embodiments, the processing core of the MCU can be operable to receive and process the Q signal, shown as S3, in order to determine the units of rotation based on the number of rises C or toggled to set in the Q signal, which may be stored in memory. In addition to, or alternative to, the units of rotation may also be determined based on the number of falling edges or toggled to reset in the Q signal, which may be then stored in memory. The dose counts may be stored in memory by the processing core prior to the step of determining the units of rotation. The not-Q signal may be used as a contingent signal, providing the control system with redundancy functionality in case the Q signal's expected pattern fails to be demonstrated. In other embodiments, the not-Q signal may be disregarded if sent to the processing core or may be omitted from the processing core. The system may only require one GPIO input. The latch circuit 2104 can, for example, advantageously result in each unit being counted once. As another exemplary advantage, the system 2100 can be configured to avoid repeat dose counts if contact arm contacts same pad on next dosing.

Another exemplary advantage of system 2100 is that the system 2100 can act as a robust debounce circuit. That is, if there is a non-uniform signal coming into the latch circuit 2104, because of the latching functionality, if signal S1 is seen repeatedly, there will be no state change, as this will only occur once there is a signal from S2. The techniques can provide advantages over systems that simply use a debounce circuit and/or software debounce. For example, the amount of debounce a non-uniform signal might need can be dependent on the frequency of the signal. Some debounce techniques add a delay to force the controller to stop for a period of time, such that the controller is not counting during the delay. If the non-uniform signal's frequency is high, and the debounce is set high (e.g., such that the delay used to force the controller to stop for a particular time period is greater than the signal frequency), then the multiple signals are blurred together and the controller determines a dose count that is less than expected. Similarly, if the non-uniform signal's frequency is low, and the debounce is set low (e.g., such that the delay used to force the controller to stop for a particular time period is shorter than the signal frequency), the controller determines a dose count that is higher than expected. Additionally, because of the presence of alternating signals, when the diameter of ring of teeth is small, the systems described herein may improve upon the mechanical tolerances of the system. For example, with only one signal, the position indicator might maintain contact and keep the switch closed when moving between teeth, and the controller may not be able to detect such transitions between teeth (resulting in no count for such transitions). In another embodiment, more than one single pole, single throw (SPST) switch, such as two SPST switches, can be applied to the circuit in FIG. 21, which would replace the SPDT switch. The two SPST switch may be offset in a manner to generate a signal representative of the reset signal and another signal representative of the set signal for inputs into the latch circuit 2104.

In some embodiments, the MCU can be configured to power on or wake up the system from a lower power state to a higher power state based on receiving the first count from the single input signal (e.g., Q). When the system is in the low power state configuration, the associated hardware and/or logic (e.g., the counter block, as described herein) has sufficient power to determine at least the initial one of a number of units.

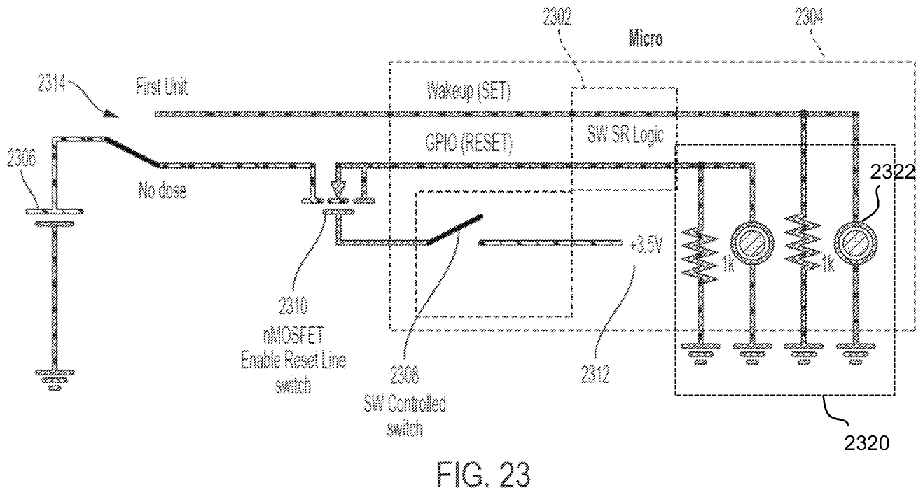

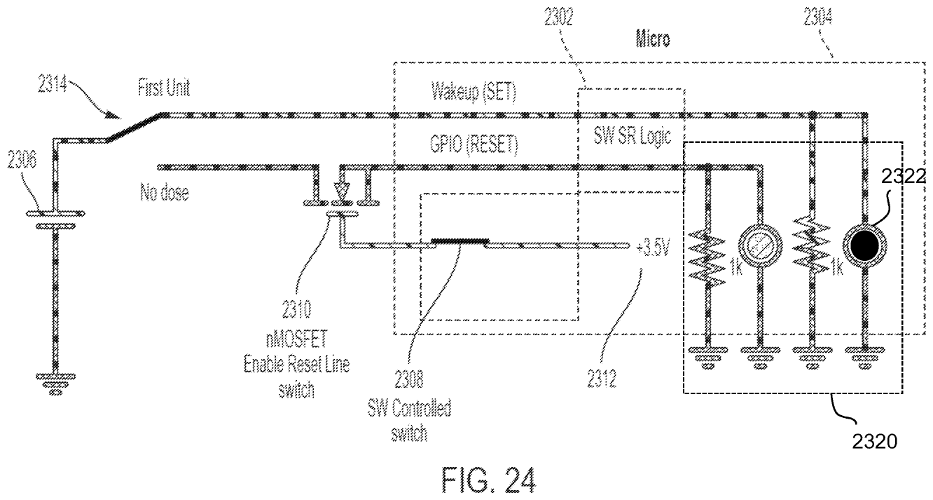

According to some embodiments, the microprocessor can implement SR logic in firmware as shown in FIGS. 23-24, according to some embodiments. FIGS. 23-24 show software (SW) SR logic 2302 within the microprocessor 2304. According to some embodiments, the microprocessor 2304 can use a MOSFET 2310 to prevent consumption of power from the battery 2306 when the medication delivery device is in a sleep state (e.g., a state prior to a first injection). For example, an initial sleep state can occur when a switch rocker of the SPDT switch is in a valley between mechanical teeth as discussed herein. As shown in FIG. 23, in the sleep state, the switch 2314, embodied as any one of the rotation sensors described herein, is in a first state that applies the voltage from the battery 2306 to the MOSFET 2310. The SW controlled switch 2308 is open so that no voltage from the voltage source 2312 is applied to the MOSFET 2310, resulting in the MOSFET 2310 being in an OFF position and therefore not conducting. Thus, in the off position, minimal and/or no current is consumed from the battery 2306. FIGS. 23 and 24 include a generic circuit portion 2320 showing a resistive load on GPIO input and indicators to show the logical state, such that the indicator 2322 shows a first state in FIG. 23 and a second state in FIG. 24. According to some embodiments, the generic circuit portion 2320 can provide signals into detection or counting functions or circuitry (e.g., a counter block), as discussed further herein.

When a dose is injected using the medication delivery device, the circuit can be configured to detect the dose injection and wake up the SR logic. For example, the circuit can be configured to detect the dose by detecting when the rocker arm of the SPDT switch crosses a peak of the mechanical teeth as discussed herein. As shown in FIG. 24, when the dose administration is detected, the switch 2314 changes to a second state that sends a wakeup signal to the microprocessor 2304 for full debounce operation of the software SR logic 2302. Additionally, the SW controlled switch 2308 is closed to apply a voltage from the voltage source 2312 to enable the MOSFET 2310 to provide a GPIO reset signal to the SW SR logic 2302. Although embodiments here describe the use of a power switch embodied as a SW controlled switch 2308, the power switch may comprise other switches such as contact sensors.

In some embodiments, a counter block may be disposed between the sensor (e.g., including the conversion control module) and the processing core of the MCU and/or be implemented by the processing core. In some embodiments, the counter block can be configured to determine the number of units by counting and logging the number of rising edges and/or falling edges of the single input signal, such as the number times to toggle to set or rises C of the Q signal in FIG. 22. The total number of rising edges in the generated signal S3 is correlated to a total number of units, which is representative of an amount of rotation of the dose member. In some embodiments, the counter block may count both of the rising edges and the falling edges to make an amount of rotation determination. The counter block can be operable to communicate the determined total number of counts C to the MCU.

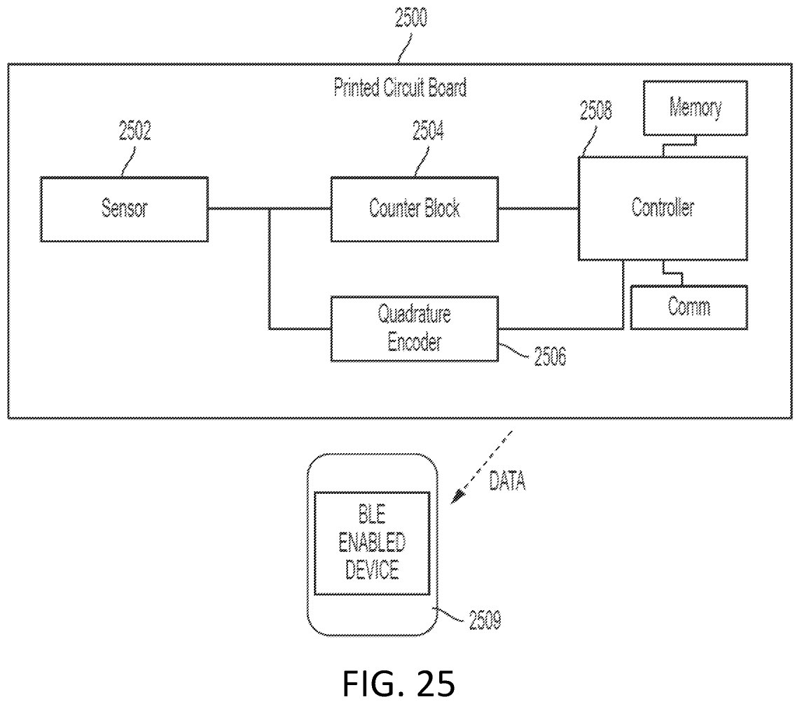

FIG. 25 is an exemplary block diagram pictorially illustrating functional aspects of a printed circuit board 2500 for processing signals from a sensor 2502 (e.g., a SPDT switch), according to some embodiments. The sensor 2502 is in communication with both the counter block 2504 and the quadrature encoder 2506, which are both in communication with the controller 2508. The sensor 2502 can be, for example, the sensor 86 in FIG. 6. For example, as described herein the sensor 86 may be configured to track the movement of the contact surface 111 and associate the movement with an output signal that is sent to the counter block 2504 and the quadrature encoder 2506, which can count the number of times the contact surface 111 slides into and out of the recesses 124 defined between the teeth 102 of the flange 38, and provide the determined count to the controller 2508 (which can be used to determine the dose of an injection). It should be appreciated that the quadrature encoder 2506 can be implemented in firmware, in hardware, and/or the like.

In some embodiments, the counter block 2504 and the quadrature encoder 2506 can be configured to process signals from the sensor 2502 using different techniques. Using different techniques can provide for some redundancy in measuring signals from the sensor 2502 (e.g., to gauge whether the measurements are accurate). Referring further to FIG. 22, FIG. 22 shows exemplary signals 2202, 2204, and part of 2206, each of which is a square wave in this example (where the horizontal axis represents time, and the vertical axis represents voltage). The counter block 2504 can analyze the received signals using a first technique to generate a first count of the signals. For example, the counter block 2504 can be configured to count the rising edges C of each square wave 2202-2206. The quadrature encoder 2506 can analyze the received signals using a second technique to generate a second count of the signals. For example, the quadrature encoder 2506 can be configured to count both the rising edges and falling edges of each square wave 2202-2206, one of the falling edges being labelled for illustrative purposes as the falling edge 2202B of the square wave 2202.

In some embodiments, the counter block 2504 and the quadrature encoder 2506 can be configured to process the signal from the sensor 2502 using different sampling rates. For example, the counter block 2504 can have a sample rate configured to sample the signal from the sensor 2502 to sense the rising edge of each signal, and the quadrature encoder 2506 can have a different sampling rate configured to sample the signal from the sensor 2502 to sense both the rising and falling edges of each signal. As an illustrative example, the counter block 2504 can be configured to use a 10 ms (100 Hz) sampling rate, while the quadrature encoder 2506 can be configured to use a 1 ms (1000 Hz) sampling rate.

The shown device is a reusable pen-shaped medication injection device, generally designated, which is manually handled by a user to selectively set a dose and then to inject that set dose. Injection devices of this type are well known, and the description of the device is merely illustrative, as the sensing system can be adapted for use in variously configured medication delivery devices, including differently constructed pen-shaped medication injection devices, differently shaped injection devices, and infusion pump devices. The medication may be any of a type that may be delivered by such a medication delivery device. The device is intended to be illustrative and not limiting as the sensing system described herein may be used in other differently configured devices.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

Various aspects are described in this disclosure, which include, but are not limited to, the following aspects:

1. A medication delivery device comprising: a housing; a mechanical switch mounted to a printed circuit board, wherein the mechanical switch comprises a single-pole double-throw (SPDT) switch comprising an arm; a rotatable element that is rotatable relative to the printed circuit board, the rotatable element having a series of protrusions that are spaced from one another, the rotatable element being positioned to permit the protrusions to slide against the arm of the SPDT switch; a conversion control module in electrical communication with the SPDT switch configured to generate an undulating unit signal based on signals from the SPDT switch as the arm slides against the protrusions; and a controller configured to receive the undulating unit signal from the conversion control module.

2. The medication delivery device of aspect 1, wherein: the SPDT switch comprises a set state that generates a set signal and a reset state that generates a reset signal; and the conversion control module comprises SR logic configured to generate the undulating unit signal based on the set and reset signals from the SPDT switch.

3. The medication delivery device of aspect 2, wherein the medication delivery device comprises a counter block configured to determine a number of units of rotation of the rotatable element based on a number of rising edges, falling edges, or both of the generated undulating unit signal.

4. The medication delivery device of any one of aspects 1-3, further comprising: a battery; and a microprocessor.

5. The medication delivery device of aspect 4, wherein the microprocessor comprises one or more of the SR logic and the controller.

6. The medication delivery device of aspect 4, further comprising a metal-oxide-semiconductor field-effect transistor (MOSFET), wherein the SPDT switch is in electrical communication with the battery.

7. The medication delivery device of aspect 6, wherein the microprocessor comprises a software (SW) controlled switch disposed between a voltage source and the MOSFET, wherein: in a sleep state, the SPDT switch is in a first state at which the battery is in electrical communication with the MOSFET, and the SW controlled switch is open so that the MOSFET is not in electrical communication with the voltage source to prevent battery drainage; and in a wakeup state, the SW controlled switch is closed so that the MOSFET is in electrical communication with the voltage source.

8. The medication delivery device of aspect 7, wherein the microprocessor comprises a reset input and a wakeup input.

9. The medication delivery device of aspect 8, wherein in the wakeup state: the MOSFET applies a voltage to the reset input; and the SPDT switch is in a second state at which the battery is in electrical communication with the wakeup input to the microprocessor.

10. The medication delivery device of any one of aspects 1-9, further comprising: an outlet; and a dose button that is axially translatable relative to the housing to activate a dose dispensing mode in which medication is dispensed out of the outlet, the dose button being rotatable relative to the housing in a dose setting mode to select a medication dose size to be delivered out of the outlet.

11. The medication delivery device of aspect 10, wherein the rotatable element is positioned to permit the protrusions to slide against the arm of the SPDT switch to move the arm among a set state and a reset state of the SPDT switch as the rotatable element rotates.

12. The medication delivery device of aspect 10, wherein the rotatable element is rotatable with the dose button in the dose setting mode and rotatable relative to the dose button in the dose dispensing mode, wherein a degree of rotation of the rotatable element during the dose dispensing mode determines an amount of medication to be dispensed out of the outlet.

13. The medication delivery device of aspect 11, wherein the SPDT switch is configured to sense rotation of the rotatable element relative to the dose button.

14. The medication delivery device of any one of aspects 1-13, wherein the printed circuit board is fixed to the dose button.

15. The medication delivery device of any one of aspects 1-14, wherein the mechanical switch comprises a base connected to an arm of the SPDT switch, the base being mounted to the printed circuit board.

16. The medication delivery device of any one of aspects 1-15, wherein the housing comprises a reservoir and a medication within the reservoir.

17. A dose detection system for a medication delivery device, comprising: a mechanical switch mounted to a printed circuit board, wherein the mechanical switch comprises a single-pole double-throw (SPDT) switch comprising an arm; a rotatable element that is rotatable relative to the printed circuit board, the rotatable element having a series of protrusions that are spaced from one another, the rotatable element being positioned to permit the protrusions to slide against the arm of the SPDT switch; a conversion control module in electrical communication with the SPDT switch configured to generate an undulating unit signal based on signals from the SPDT switch as the arm slides against the protrusions; and a controller configured to receive the undulating unit signal from the conversion control module.

18. The dose detection system of aspect 17, wherein: the SPDT switch comprises a set state that generates a set signal and a reset state that generates a reset signal; and

the conversion control module comprises SR logic configured to generate the undulating unit signal based on the set and reset signals from the SPDT switch.

19. The dose detection system of aspect 18, wherein the medication delivery device comprises a counter block configured to determine a number of units of rotation of the rotatable element based on a number of rising edges, falling edges, or both of the generated undulating unit signal.

20. The dose detection system of aspect 19, further comprising: a battery in electrical communication with the SPDT switch; a metal-oxide-semiconductor field-effect transistor (MOSFET); and a microprocessor, wherein the microprocessor comprises one or more of the SR logic and the controller.

21. The dose detection system of aspect 20, wherein the microprocessor comprises a software (SW) controlled switch disposed between a voltage source and the MOSFET, wherein: in a sleep state, the SPDT switch is in a first state at which the battery is in electrical communication with the MOSFET, and the SW controlled switch is open so that the MOSFET is not in electrical communication with the voltage source to prevent battery drainage; and in a wakeup state, the SW controlled switch is closed so that the MOSFET is in electrical communication with the voltage source.

22. The dose detection system of aspect 21, wherein the microprocessor comprises a reset input and a wakeup input.

23. The dose detection system of aspect 22, wherein in the wakeup state: the MOSFET applies a voltage to the reset input; and the SPDT switch is in a second state at which the battery is in electrical communication with the wakeup input to the microprocessor.

24. A method comprising: rotating a rotatable element relative to a printed circuit board, the rotatable element having a series of protrusions that are spaced from one another, the rotatable element being positioned to permit the protrusions to slide against an arm of a single-pole double-throw (SPDT) switch of a mechanical switch mounted to the printed circuit board; and generating an undulating signal via a conversion control module that is in electrical communication with the SPDT switch based on signals from the SPDT switch as the arm slides against the protrusions.

25. The method of aspect 24 comprising: generating a set signal and/or a reset signal via the SPDT switch, and wherein the generating the undulating signal step further comprises generating the undulating unit signal via SR logic of the conversion control module based on the set and reset signals from the SPDT switch.

26. The method of aspect 25 comprising: determining a number of units of rotation of the rotatable element via a counter block based on a number of rising edges, falling edges, or both of the generated undulating unit signal.

27. The method of aspect 26 comprising: switching the SPDT switch to a first state at which a battery that is in electrical communication with a metal-oxide-semiconductor field-effect transistor (MOSFET); and switching a software (SW) controlled switch of a microprocessor to open so that the MOSFET is not in electrical communication with a voltage source to prevent battery drainage and define a sleep state, or switching the SW controlled switch to close so that the MOSFET is in electrical communication with the voltage source to define a wakeup state.

28. The method of aspect 27, wherein the switching the SW controlled switch to close step further comprises applying a voltage via the MOSFET to a reset input of the microprocessor; and switching the SPDT switch to a second state at which the battery is in electrical communication with a wakeup input to the microprocessor.

1. A medication delivery device comprising:
a housing;
a mechanical switch mounted to a printed circuit board, wherein the mechanical switch comprises a single-pole double-throw (SPDT) switch comprising an arm;
a rotatable element that is rotatable relative to the printed circuit board, the rotatable element having a series of protrusions that are spaced from one another, the rotatable element being positioned to permit the protrusions to slide against the arm of the SPDT switch;
a conversion control module in electrical communication with the SPDT switch configured to generate an undulating unit signal based on signals from the SPDT switch as the arm slides against the protrusions; and
a controller configured to receive the undulating unit signal from the conversion control module.

2. The medication delivery device of claim 1, wherein:
the SPDT switch comprises a set state that generates a set signal and a reset state that generates a reset signal; and
the conversion control module comprises SR logic configured to generate the undulating unit signal based on the set and reset signals from the SPDT switch.

3. The medication delivery device of claim 2, wherein the medication delivery device comprises a counter block configured to determine a number of units of rotation of the rotatable element based on a number of rising edges, falling edges, or both of the generated undulating unit signal.

4. The medication delivery device of claim 1, further comprising:
a battery; and
a microprocessor.

5. The medication delivery device of claim 4, wherein the microprocessor comprises one or more of the SR logic and the controller.

6. The medication delivery device of claim 4, further comprising a metal-oxide-semiconductor field-effect transistor (MOSFET), wherein the SPDT switch is in electrical communication with the battery.

7. The medication delivery device of claim 6, wherein the microprocessor comprises a software (SW) controlled switch that is disposed between a voltage source and the MOSFET, wherein:
in a sleep state, the SPDT switch is in a first state at which the battery is in electrical communication with the MOSFET, and the SW controlled switch is open so that the MOSFET is not in electrical communication with the voltage source to prevent battery drainage; and
in a wakeup state, the SW controlled switch is closed so that the MOSFET is in electrical communication with the voltage source.

8. The medication delivery device of claim 7, wherein the microprocessor comprises a reset input and a wakeup input.

9. The medication delivery device of claim 8, wherein in the wakeup state: the MOSFET applies a voltage to the reset input; and
the SPDT switch is in a second state at which the battery is in electrical communication with the wakeup input to the microprocessor.

10. The medication delivery device of claim 1, further comprising:
an outlet; and
a dose button that is axially translatable relative to the housing to activate a dose dispensing mode in which medication is dispensed out of the outlet, the dose button being rotatable relative to the housing in a dose setting mode to select a medication dose size to be delivered out of the outlet.

11. The medication delivery device of claim 10, wherein the rotatable element is positioned to permit the protrusions to slide against the arm of the SPDT switch to move the arm among a set state and a reset state of the SPDT switch as the rotatable element rotates.

12. The medication delivery device of claim 10, wherein the rotatable element is rotatable with the dose button in the dose setting mode and rotatable relative to the dose button in the dose dispensing mode, wherein a degree of rotation of the rotatable element during the dose dispensing mode determines an amount of medication to be dispensed out of the outlet.

13. The medication delivery device of claim 11, wherein the SPDT switch is configured to sense rotation of the rotatable element relative to the dose button.

14. The medication delivery device of claim 10, wherein the printed circuit board is fixed to the dose button.

15. The medication delivery device of claim 1, wherein the mechanical switch comprises a base connected to an arm of the SPDT switch, the base being mounted to the printed circuit board.

16. The medication delivery device of claim 1, wherein the housing comprises a reservoir and a medication within the reservoir.

17. A dose detection system for a medication delivery device, comprising:
a mechanical switch mounted to a printed circuit board, wherein the mechanical switch comprises a single-pole double-throw (SPDT) switch comprising an arm;
a rotatable element that is rotatable relative to the printed circuit board, the rotatable element having a series of protrusions that are spaced from one another, the rotatable element being positioned to permit the protrusions to slide against the arm of the SPDT switch;
a conversion control module in electrical communication with the SPDT switch configured to generate an undulating unit signal based on signals from the SPDT switch as the arm slides against the protrusions; and
a controller configured to receive the undulating unit signal from the conversion control module.

18. The dose detection system of claim 17, wherein:
the SPDT switch comprises a set state that generates a set signal and a reset state that generates a reset signal; and
the conversion control module comprises SR logic configured to generate the undulating unit signal based on the set and reset signals from the SPDT switch.

19. The dose detection system of claim 18, wherein the medication delivery device comprises a counter block configured to determine a number of units of rotation of the rotatable element based on a number of rising edges, falling edges, or both of the generated undulating unit signal.

20. The dose detection system of claim 19, further comprising:
a battery in electrical communication with the SPDT switch;
a metal-oxide-semiconductor field-effect transistor (MOSFET); and
a microprocessor, wherein the microprocessor comprises one or more of the SR logic and the controller.

21. The dose detection system of claim 20, wherein the microprocessor comprises a software (SW) controlled switch disposed between a voltage source and the MOSFET, wherein:
in a sleep state, the SPDT switch is in a first state at which the battery is in electrical communication with the MOSFET, and the SW controlled switch is open so that the MOSFET is not in electrical communication with the voltage source to prevent battery drainage; and
in a wakeup state, the SW controlled switch is closed so that the MOSFET is in electrical communication with the voltage source.

22. The dose detection system of claim 21, wherein the microprocessor comprises a reset input and a wakeup input.

23. The dose detection system of claim 22, wherein in the wakeup state:
the MOSFET applies a voltage to the reset input; and
the SPDT switch is in a second state at which the battery is in electrical communication with the wakeup input to the microprocessor.

24. A method comprising:
rotating a rotatable element relative to a printed circuit board, the rotatable element having a series of protrusions that are spaced from one another, the rotatable element being positioned to permit the protrusions to slide against an arm of a single-pole double-throw (SPDT) switch of a mechanical switch mounted to the printed circuit board; and
generating an undulating signal via a conversion control module that is in electrical communication with the SPDT switch based on signals from the SPDT switch as the arm slides against the protrusions.

25. The method of claim 24 comprising:
generating a set signal and/or a reset signal via the SPDT switch, and wherein the generating the undulating signal step further comprises generating the undulating unit signal via SR logic of the conversion control module based on the set and reset signals from the SPDT switch.

26. The method of claim 25 comprising:
determining a number of units of rotation of the rotatable element via a counter block based on a number of rising edges, falling edges, or both of the generated undulating unit signal.

27. The method of claim 26 comprising:
switching the SPDT switch to a first state at which a battery that is in electrical communication with a metal-oxide-semiconductor field-effect transistor (MOSFET); and
switching a software (SW) controlled switch of a microprocessor to open so that the MOSFET is not in electrical communication with a voltage source to prevent battery drainage and define a sleep state, or switching the SW controlled switch to close so that the MOSFET is in electrical communication with the voltage source to define a wakeup state.

28. The method of claim 27, wherein the switching the SW controlled switch to close step further comprises applying a voltage via the MOSFET to a reset input of the microprocessor; and switching the SPDT switch to a second state at which the battery is in electrical communication with a wakeup input to the microprocessor.
